(19) United States
(12) Patent Application Publication
(10) Pub. No.: US 2014/0276053 A1
(43) Pub. Date: Sep. 18, 2014

(54) MULTI-ELEMENT THERAPY AND IMAGING TRANSDUCER FOR ULTRASOUND THERAPY

(71) Applicant: SonaCare Medical, LLC, Charlotte, NC (US)

(72) Inventors: Narendra Sanghvi, Indianapolis, IN (US); Adam Morris, Camden, IN (US)

(73) Assignee: SonaCare Medical, LLC, Charlotte, NC (US)

(21) Appl. No.: 14/212,352

(22) Filed: Mar. 14, 2014

Related U.S. Application Data
(60) Provisional application No. 61/782,471, filed on Mar. 14, 2013.

(51) Int. Cl. A61B 8/00; A61B 1/06; A61B 1/05; A61N 7/00; A61B 8/12

(52) U.S. Cl. CPC A61B 8/4494; A61N 7/00; A61B 8/12; A61B 1/05; A61B 1/0676; A61B 8/546; A61N 2007/0052
USPC 600/439

(57) ABSTRACT

A transducer assembly for providing ultrasound may include a shaft operatively connected to a motor. A housing may include a receptacle for receipt of at least a portion of the shaft therein, two spaced-apart therapy elements, and an imaging element positioned between the therapy elements. The imaging element and the therapy elements may be spatially oriented in a confocal beam configuration. The assembly may be sized and shaped to pass through a trocar port.

### CROSS-REFERENCE TO RELATED APPLICATIONS

The present application claims priority to U.S. Provisional Application No. 61/782,471, entitled “MULTI-ELEMENT CURVED RECTANGULAR/ELLIPTICAL/CYLINDRICAL THERAPY AND IMAGING TRANSDUCER FOR HIFU THERAPY WITH ULTRASOUND IMAGE GUIDANCE” and filed on Mar. 14, 2013, which is herein incorporated by reference in its entirety.

### BACKGROUND

Among the technologies being considered, developed or currently deployed for use in treating abnormalities of human and animal tissue is focused ultrasound (FU). Focused ultrasound devices use ultrasound transducers to deliver generally thermal or cavitational dose to a small, well-defined spot at some fixed or focal distance from the transducer surface. Typically, the region to be treated is larger than the small spot of dose that is delivered from the transducer.

One way to a deliver thermal dose to a larger region is to move the transducer so that the small spot of thermal dose is scanned over the region that is to receive thermal or cavitational dose. Another way is to move the patient relative to the transducer. The latter approach is often used in extracorporeal devices where the transducer is located outside the patient. Such is the case with the EXABLATE system. The former approach is used often in devices where the transducer is located inside the patient. Such is the case with devices such as the Sonatherm and SONABLATE devices.

In devices where the transducer is introduced into the patient and is moved potentially relative to the patient, it is typically deployed in a probe. Typically, such probes include an acoustic window for passage of the FU and a way to coupling the acoustic window to the tissue to be treated. Coupling involves providing a continuous acoustic path between the transducer and the tissue being treated. The coupling mechanism, typically degassed water, is contained by an acoustically invisible membrane made typically of a material, such as latex. The acoustic window, through which the FU will pass, and its coupling mechanism are brought in contact with the tissue through which the FU will pass. In instances where the probe is introduced into a sterile environment, the portion of the probe that comes in contact with tissue should be sterile. The water used to provide the coupling mechanism also should be sterile. The need for sterile coupling may require a tip or cover to be placed over the FU probe, thereby adding to the size of the device used to deliver FU.

It is often beneficial to perform ultrasound (US) imaging in conjunction with the FU therapy transducer to localize the region to which the FU will be delivered. The US imaging transducer can be separate from the FU therapy transducer, deployed either as an independent probe or as a separate component of the FU probe. Alternatively, it can be integrated into the FU transducer, allowing a single transducer and probe to image and treat with US. An example of such a design is taught in U.S. Pat. No. 8,038,631 (“the '631 patent), which is herein incorporated by reference, as deployed in a transducer with a diameter of 18 mm or greater. The '631 patent teaches a curved rectangular transducer designed for therapy that has as an integrated center element for imaging. A mechanically scored grove on the ceramic provides an electrical isolation between imaging and treatment crystals (U.S. Pat. No. 5,117,832), both of which are made out of a same ceramic substrate operating at the same frequency of 4 MHz. The focal intensity (Watts/cm²) of the therapy portion of the transducer is designed to be sufficient to produce tissue necrosis in kidney and other highly perfused organs.

Such integrated imaging and therapy transducers and probes can be used in a number of clinical situations; each clinical use may have specific design constraints. Some of these constraints for high-intensity focused ultrasound (HIFU) transducers are described in U.S. Pat. No. 5,117,832 specifically for use in intraluminal and intracavity applications. The transducer can be placed in a close proximity to the targeted organ via natural orifice of the body or via a laparoscopic port. However, this requirement poses constraints on the size and shape of the applicator and transducer in terms of acoustic power generation and required beam formation and steering. For instance, ports of various sizes are used commonly in laparoscopic surgery for treatment of abdominal and pelvic malignancies. The entire probe plus any protective covering used to insure sterility of the system must pass through the port. The transducer itself needs to be smaller, yet still capable of delivering the power required to destroy targeted tissue.

A transducer with such a small diameter can be difficult to achieve using the typical transducer design. If manufactured as one piece, the piezo element will be extremely fragile due to the thin and narrow cross-section between the imaging element in the center and the therapy elements on either side. If manufactured as multiple pieces, the problem is insuring that all elements align correctly.

Another issue with currently available designs of integrated transducers is that HIFU generally uses a low frequency and a narrow bandwidth in order to provide deep tissue penetration without excessive tissue temperature elevation in the near field. Ultrasound imaging, on the other hand, benefits from a higher frequency and wider bandwidth in order to achieve higher spatial and contrast resolution.

### BRIEF SUMMARY

Therefore, there is a need for a new transducer design when imaging and therapy are to be delivered in a single transducer and where the transducer will be required to pass through small orifices that can achieve optimal design requirements for both imaging and therapy.

According to one embodiment of the present disclosure, a transducer design is provided for an integrated imaging and therapy ultrasound transducer that includes multiple spherical or cylindrical crystals formed from the surface of a spherical disk that can be manufactured in sizes small enough so that it can pass through the surgical ports in use commonly and can deliver the acoustical power and frequencies required to both image and ablate target tissue.

In an embodiment of the present disclosure, the transducer design is such that it can be manufactured using standard US crystals in a size that is small enough so that it and its associated coverings and protective enclosure can pass through a surgical robotic port.

In an embodiment of the present disclosure, the therapy portion of the transducer will provide total acoustic power of 30 Watts and generate minimum focal intensity in tissue, Iₛₚₜₐ (spatial peak, temporal average), over 500 W/cm² to induce coagulative necrosis in the targeted tissue.

In an embodiment of the present disclosure, the alignment of confocal beams from the one imaging and two separate “split beam” therapy elements is accomplished through mechanical alignment control of mounting surfaces within a transducer housing.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings various illustrative embodiments. It should be understood, however, that the invention is not limited to the precise arrangements and instrumentalities shown. In the drawings:

FIG. 1 shows a spherical crystal with four identical segments according to an embodiment of the present disclosure, wherein the segments provide multiple elements (shaded) with identical size and same radius of curvature for transducer fabrication;

FIG. 2 is a top plan view of a transducer assembly according to an embodiment of the present disclosure;

FIG. 3a is a graph displaying dielectric loss factor for EC-69, EC-64 and EC-65 ceramics;

FIG. 3b is a graph displaying dielectric loss factor for EC-97 ceramic;

FIG. 4 is an elevation view of a transducer assembly according to an embodiment of the present disclosure;

FIG. 5 is a perspective view of transducer assembly and a solid transducer shaft joint according to an embodiment of the present disclosure, wherein the joint provides two degrees of freedom for beam steering;

FIG. 6 is a parameters chart or list to stimulate lesion size and shape;

FIG. 7 is a perspective view of a simulated transducer assembly according to an embodiment of the present disclosure;

FIG. 8 shows a trajectory path for transducer x-y movement during treatment to provide sufficient ultrasound energy dose and assure complete coverage of the therapeutic target; and

FIG. 9 shows a simulated lesion produced at 8 minutes, wherein the size of the lesion is at 20 cubic centimeters (CC).

### DETAILED DESCRIPTION OF THE PRESENT DISCLOSURE

Various embodiments of the present disclosure are described hereinafter with reference to the figures. It should be noted that the figures are not drawn to scale and elements of similar structures or functions are represented by like reference numerals throughout the figures. It should also be noted that the figures are not intended to facilitate the description of specific embodiments of the present disclosure. The figures are not intended as an exhaustive depiction or description of the present disclosure, or a limitation on the scope of the present disclosure. In addition, an aspect described in conjunction with a particular embodiment of the present disclosure is not necessarily limited to that embodiment and may be practiced in any other embodiments of the present disclosure. It will be appreciated that while various embodiments of the present disclosure are described in connection with radiation treatment of tumors, the claimed disclosure has application in other industries and to targets other than cancers.

According to an embodiment of the present disclosure, one or more ultrasound transducer 10 may be formed from multiple spherical or cylindrical crystals or piezo elements, as understood by those skilled in the art, fabricated from the surface of a spherical disk, as shown in FIG. 1. The shaded segments shown in FIG. 1 are portions cut from the spherical disk in order to create the parts used to assemble the transducer 10, as described in detail below. The crystals may provide the total acoustic power of 30 Watts and minimum focal intensity in tissue, Iₛₚₜₐ (spatial peak, temporal average), of 500 W/cm² required to induce coagulative necrosis in the targeted tissue (see FIGS. 8 and 9).

The size, shape and/or number of elements of the transducer 10 may selected based on the particular application. As shown in FIGS. 2, 4, 5 and 7, a transducer 10 according to one or more embodiments of the present disclosure may be constructed from three or more elements, as shown in FIG. 2. For example, at least two spaced-apart outer elements 12 may be used for therapy and at least one center element 14 may be used for tissue visualization or imaging. The therapy elements 12 may operate at a selected frequency (such as at exactly or approximately 4 MHz) and have total focal gain to overcome ultrasound absorption rate in tissue and produce required Iₛₚₜₐ for tissue necrosis. One or more of the crystals shown in FIG. 1 may form the therapy elements 12. The imaging element 14 may operate at exactly or approximately 6.5 MHz, for example, which provides high resolution imaging of the tissue targeted for treatment. The imaging and therapy elements 14, 12 may be spatially oriented in confocal beam configuration so that tissue imaged prior to treatment, during treatment and imaged for tissue change monitoring (during treatment) are always in registration. One or more thermistors 16 or other objects, such as a camera, a light (e.g., LED) and/or a transducer or the like, may be positioned and/or integrated in a distal end of a housing 18 of the transducer 10. In an embodiment including the thermistor 16, the thermistor 16 may monitor liquid or water bath temperature.

The transducer 10 may be sized such that it and its associated coverings and protective materials will pass through a required device. In one non-limiting example, when using a conventional trocar with a port having a diameter of exactly or approximately 12 millimeters (mm), the transducer 10 must be no more than 8 mm in diameter. It can also be used for intraluminal application via a catheter to pass through standard 22-28 fr. Endoscopes and/or resectoscope. In such applications, the transducer may need to be as small as 6-7 mm in diameter.

In order to effectively deliver the acoustic power of 30 W, the power input density capacity per cubic meter, and the power dissipation capacity per cubic meter, P_{d}, of the piezoelectric material must be known. The power density capacity is given by the following equation:

Pᵢ=E²ωkₜ²Qₘ∈^{T}₃₃

The power dissipation capacity is given by the following equation:

P_{d}=E²ω∈^{T}₃₃ tan δ,

where “E” is the electric field in rms voltage per unit thickness, “ω” is the radian frequency, 2πf, “kₜ” is the thickness coupling factor, “Qₘ” is the thickness mechanical quality factor, “∈” is the dielectric constant, and “δ” is the dielectric loss.

For power density, only the dielectric constant (∈) need be considered as all other parameters can be considered constant when operating at frequencies in the MHz range. The dielectric constant (∈) may be chosen such that an electrical impedance match can be made as close as possible to the electrical power source. This minimizes losses in the electrical system due to power reflection (VSWR). The goal is to match electrical impedances in the system in order to maximize system efficiency.

On the other hand, once the dielectric is specified, then the power dissipation within the ceramic has to be evaluated. Since the volume of piezoelectric ceramic is small, the power dissipation capacity must be maximized, with or without some form of cooling. As electrical power is applied and increased to the level required to obtain 30 W of acoustic power output, the piezoelectric ceramic will heat up due to various loss mechanisms. It is important that the dielectric loss remain stable and low as the electrical field increases. FIGS. 3a and 3b graph dielectric loss factor for EC-69 (PZT-8 Navy Type III), EC-64 (PZT-4 Navy Type I) and EC-97 (Lead Titanate), which are examples of high power piezoelectric ceramic compositions that have similar properties and maintain relatively low dielectric loss as electric field is increased. There are a few other related PZT compositions such as PZT-2 and PZT-4D as well as several barium titanate compositions and potentially quartz that could be utilized for similar designs.

By manipulating the equation for input power capacity, the limit for input power per square meter of surface area can be obtained as follows:

Pᵢ/M²=tω∈k²E²Q,

where “t” is thickness in meters. The maximum Pᵢ is generally stated in the 50-75 W/cm² range. For the current design, the width and total length of the radiating piezoelectric ceramic element may be 0.745 cm and 2.3 cm, respectively and the total surface area may be 1.71 cm². At the maximum input power density, this implies a maximum input power of 128 W. This should be balanced with the maximum allowable electrical field applied across the thickness of the element without causing excessive electrical dissipation or heat build-up. Referring FIGS. 3a and 3b, this is between 4000 and 8000 V/cm. The elements are about 0.051 cm thick; therefore the maximum applied voltage would be about 408 Vrms and since input power is also described as follows:

Pᵢ=V²/R

And assuming R, the resistance of the element, has been designed to match a source impedance of 50 Ohms, and then the maximum Pi that can be applied across the thickness is 3.3 kW. Since this number is much larger than the maximum input for the surface area of 128 W, the limit is 128 W and the other result is ignored as this would most likely cause failure thru dielectric breakdown or mechanical fatigue and fracture.

Assuming a worst case of 30% efficiency to obtain 30 W of acoustic output, then the input power required is 90 W, well within both limitations.

In order to manufacture the transformer 10 described above so that it can be used correctly, the imaging and therapy elements 14, 12 may be confocal. Referring to FIG. 4, the alignment of confocal beams of imaging and two separate “split beam” therapy elements 14, 12 may be accomplished through mechanical alignment control of mounting surfaces, pockets or set back lips are formed on or within the housing 18 of the transducer 10. The mounting surfaces or pockets may simply be one or more portions of the housing 10 in which the crystals sit or are located. The pockets are provided for aligning the crystals in the lateral planes such that the therapy elements 12 are confocal in three (3) planes. The pockets are designed such that they set the spacing between the therapy elements 12 along the contour of a sphere with the imaging element 14 located at the geometric center of the therapy elements 12.

In at least one embodiment of the present disclosure, certain requirements for constructing the transducer 10 may exist if the transducer 10 is to operate as specified above. For example, at least a portion of the pocket, such as an internal surface thereof, may be spherical with a convex radius equal to that of the concave surface of the crystal(s) or piezo element(s). The pocket or lip allows the crystal(s) to sit flush or even with an exterior surface of the housing 18. Alternatively, if a matching layer of sound transmittable material is used, then the crystal(s) sit low enough in the housing so that the matching layer is flush with the exterior surface of the housing 18. Thus, a depth of the pocket(s) for the therapy elements 12 may be such that the external (outer) surface of the matching layer is coplanar with the external concave surface of the housing 18. The depth of the imaging element 14 may also be set back within the housing 18 such that an outer surface of a matching layer for the three elements 12, 14, if used, is coplanar with an external concave surface of the housing 18.

The transducer 10 may be made of materials that are compatible with all gas and liquid sterilization processes, except the use of heat greater than 70 C. The material(s) used to form the transducer 10 may also be compatible with all forms of disinfection.

Referring to FIG. 5, a shaft 20 of the transducer can be keyed so that it can be directly driven with a rotational motion “B” with a rotary mechanism or motor 24. The shaft 20 also can be keyed such that it can be driven, directly or otherwise, in a linear motion “A” with a linear mechanism or motor 24. A controller 26 may effectuate operation of the motor 24, among other features of the transducer 10. As shown in FIGS. 2 and 4, a proximal end of the housing 18 of the transducer 10 includes a receptacle 22 for receiving and/or engaging at least a portion of the shaft 20, such as a distal end thereof. The housing 18 may be permanently or removably attached to the shaft 20.

It will be appreciated by those skilled in the art that changes could be made to the embodiments described above without departing from the broad inventive concept thereof. It is understood, therefore, that this disclosure is not limited to the particular embodiments disclosed, but it is intended to cover modifications within the spirit and scope of the present disclosure as defined by the appended claims.

I/We claim:

1. A transducer assembly for providing ultrasound, the assembly comprising:
a shaft operatively connected to a motor; and
a housing including a receptacle for receipt of at least a portion of the shaft therein, two spaced-apart therapy elements, and an imaging element positioned between the therapy elements, the imaging element and the therapy elements being spatially oriented in a confocal beam configuration and being configured to emit ultrasound energy to a targeted area,
wherein the assembly is sized and shaped to pass through a trocar port.

2. The assembly according to claim 1, wherein at least a portion of the assembly is formed from multiple spherical or cylindrical crystals fabricated from a surface of a spherical disk.

3. The assembly according to claim 2, wherein the spherical disk is manufactured in a size that is sufficiently small so that the assembly can pass through the trocar port and deliver acoustical power and frequencies to image and ablate target tissue.

4. The assembly according to claim 2, wherein the crystals provide a total acoustic power of 30 Watts and a minimum focal intensity in tissue of 500 W/cm² to induce coagulative necrosis in the tissue.

5. The assembly according to claim 1, wherein the therapy elements operate at a selected frequency of approximately 4 MHz and the imaging element 14 operates at approximately 6.5 MHz and provides high resolution imaging of tissue targeted for treatment.

6. The assembly according to claim 1, wherein the housing further includes at least one object at a distal end thereof, the object being selected from the group consisting of one or more cameras, lights and thermistors.

7. The assembly according to claim 6, wherein the object is at least one thermistor that monitors water temperature.

8. The assembly according to claim 1, wherein the therapy elements are comprised of multiple crystals from the surface of a spherical disk.

9. The assembly according to claim 1, wherein the motor drives the shaft in at least one of a linear motion and a rotational motion.

10. A method for treating a targeted area of tissue, the method comprising:
providing a transducer assembly sufficiently small to pass through a trocar port, the assembly including two spaced-apart therapy elements and an imaging element positioned between the therapy elements, the imaging element and the therapy elements being spatially oriented in a confocal beam configuration; and
delivering 30 W of acoustic power to a targeted area of tissue via the transducer assembly.

12. The method according to claim 11, further comprising:
calculating power density capacity (Pᵢ) as follows:
Pᵢ=E²ωkₜ²Qₘ∈^{T}₃₃,
wherein “E” is the electric field in rms voltage per unit thickness, “ω” is the radian frequency, 2πf, “kₜ” is the thickness coupling factor, “Qₘ” is the thickness mechanical quality factor, and “∈” is the dielectric constant.

13. The method according to claim 12, further comprising:
calculating power dissipation capacity (P_{d}) as follows:
P_{d}=E²ω∈^{T}₃₃ tan δ,
wherein “δ” is the dielectric loss.

14. A system for providing ultrasound comprising:
a trocar including a port;
a housing sufficiently small to pass through the port of the trocar;
a shaft having a first end operatively connected to a motor and an opposing second end operatively connected to the housing;
two spaced-apart therapy elements comprising multiple crystals from a surface of a spherical disk; and
an imaging element positioned between the two therapy elements,
wherein the imaging element and the therapy elements being spatially oriented in a confocal beam configuration,

15. The system according to claim 14, wherein the spherical disk is manufactured to a size that is sufficiently small so that the housing can pass through the port of the trocar and deliver acoustical power and frequencies to image and ablate target tissue.

16. The assembly according to claim 15, wherein the crystals provide a total acoustic power of 30 Watts and a minimum focal intensity in tissue of 500 W/cm² to induce coagulative necrosis in the tissue.
